(19) United States
(12) Patent Application Publication
(10) Pub. No.: US 2012/0046599 A1
(43) Pub. Date: Feb. 23, 2012

(54) THERAPEUTIC AGENT DELIVERY SYSTEM, DEVICE AND METHOD FOR LOCALIZED APPLICATION OF THERAPEUTIC SUBSTANCES TO A BIOLOGICAL CONDUIT

(75) Inventors: Victor Leo Schoenle, Greenfield, MN (US); Walter John Dobrovolny, St. Paul, MN (US); Rainer Schnabel, Middleton, WI (US)

(73) Assignee: CARDIOVASCULAR SYSTEMS, INC., St. Paul, MN (US)

(21) Appl. No.: 13/027,744

(22) Filed: Feb. 15, 2011

Related U.S. Application Data
(60) Provisional application No. 61/305,637, filed on Feb. 18, 2010.

(51) Int. Cl. A61M 37/00

(52) U.S. Cl. 604/22

(57) ABSTRACT

The invention provides a system, device and method for localized application of therapeutic substances within a biological conduit after the lumen wall has been scored by an eccentric scoring head. One embodiment comprises radial scoring with the eccentric scoring head, with a therapeutic agent coated balloon inflated distal to the scoring and dragged proximally through the scoring. Another embodiment comprises inflation of two anchor balloons on either side of scoring with subsequent inflation of a therapeutic agent coated balloon therebetween which causes the distance between anchor balloons to increase, thus stretching the scoring crevices while applying the agent therein with subsequent closure of crevices on deflation of anchor and application balloons. Another embodiment comprises an inflated anchor balloon with a threaded scoring device wherein the scoring members are coated with agent and rotation of the threaded device enables travel in the proximal direction away from anchor balloon.

### BACKGROUND OF THE INVENTION

1. Field of the Invention

The invention relates to systems, devices and methods for treating biological conduits, e.g., animal lumens, with localized delivery of therapeutic agents.

2. Description of the Related Art

A variety of techniques and instruments have been developed for use in the removal or repair of tissue in biological conduits, e.g., without limitation, blood vessels and similar body passageways. A frequent objective of such techniques and instruments is the removal of atherosclerotic plaques in a patient's arteries. Atherosclerosis is characterized by the buildup of fatty deposits (atheromas) in the intimal layer (under the endothelium) of a patient's blood vessels. Very often over time, what initially is deposited as relatively soft, cholesterol-rich atheromatous material hardens into a calcified atherosclerotic plaque. Such atheromas restrict the flow of blood, and therefore often are referred to as stenotic lesions or stenoses, the blocking material being referred to as stenotic material. If left untreated, such stenoses can cause angina, hypertension, myocardial infarction, strokes, leg pain and the like.

Rotational atherectomy procedures have become a common technique for removing such stenotic material. Such procedures are used most frequently to initiate the opening of calcified lesions in coronary arteries. Most often the rotational atherectomy procedure is not used alone, but is followed by a balloon angioplasty procedure, which, in turn, is very frequently followed by placement of a stent to assist in maintaining patency of the opened artery. For non-calcified lesions, balloon angioplasty most often is used alone to open the artery, and stents often are placed to maintain patency of the opened artery. Studies have shown, however, that a significant percentage of patients who have undergone balloon angioplasty and had a stent placed in an artery experience stent restenosis—i.e., blockage of the stent which most frequently develops over a period of time as a result of excessive growth of scar tissue within the stent. In such situations an atherectomy procedure is the preferred procedure to remove the excessive scar tissue from the stent (balloon angioplasty being not very effective within the stent), thereby restoring the patency of the artery.

Several kinds of rotational atherectomy devices have been developed for attempting to remove stenotic material. In one type of device, such as that shown in U.S. Pat. No. 4,990,134 (Auth), a burr covered with an abrasive abrading material such as diamond particles is carried at the distal end of a flexible drive shaft. The burr is rotated at high speeds (typically, e.g., in the range of about 150,000-190,000 rpm) while it is advanced across the stenosis. As the burr is removing stenotic tissue, however, it blocks blood flow. Once the burr has been advanced across the stenosis, the artery will have been opened to a diameter equal to or only slightly larger than the maximum outer diameter of the burr. Frequently more than one size burr must be utilized to open an artery to the desired diameter.

U.S. Pat. No. 5,314,438 (Shturman) discloses another atherectomy device having a drive shaft with a section of the drive shaft having an enlarged diameter, at least a segment of this enlarged surface being covered with an abrasive material to define an abrasive segment of the drive shaft. When rotated at high speeds, the abrasive segment is capable of removing stenotic tissue from an artery. Though this atherectomy device possesses certain advantages over the Auth device due to its flexibility, it also is capable only of opening an artery to a diameter about equal to the diameter of the enlarged abrading surface of the drive shaft since the device is not eccentric in nature.

U.S. Pat. No. 6,494,890 (Shturman) discloses an atherectomy device having a drive shaft with an enlarged eccentric section, wherein at least a segment of this enlarged section is covered with an abrasive material. When rotated at high speeds, the abrasive segment is capable of removing stenotic tissue from an artery. The device is capable of opening an artery to a diameter that is larger than the resting diameter of the enlarged eccentric section due, in part, to the orbital rotational motion during high speed operation. Since the enlarged eccentric section comprises drive shaft wires that are not bound together, the enlarged eccentric section of the drive shaft may flex during placement within the stenosis or during high speed operation. This flexion allows for a larger diameter opening during high speed operation, but may also provide less control than desired over the diameter of the artery actually abraded. In addition, some stenotic tissue may block the passageway so completely that the Shturman device cannot be placed therethrough. Since Shturman requires that the enlarged eccentric section of the drive shaft be placed within the stenotic tissue to achieve abrasion, it will be less effective in cases where the enlarged eccentric section is prevented from moving into the stenosis. The disclosure of U.S. Pat. No. 6,494,890 is hereby incorporated by reference in its entirety.

U.S. Pat No. 5,681,336 (Clement) provides an eccentric tissue removing burr with a coating of abrasive particles secured to a portion of its outer surface by a suitable binding material. This construction is limited, however because, as Clement explains at Col. 3, lines 53-55, that the asymmetrical burr is rotated at “lower speeds than are used with high speed ablation devices, to compensate for heat or imbalance.” That is, given both the size and mass of the solid burr, it is infeasible to rotate the burr at the high speeds used during atherectomy procedures, i.e., 20,000-200,000 rpm. Essentially, the center of mass offset from the rotational axis of the drive shaft would result in development of significant centrifugal force, exerting too much pressure on the wall of the artery and creating too much heat and excessively large particles.

Another method of treatment of occluded vessels may include the use of stents. Stents may be placed at the site of a stenosis and expanded to widen the vessel, remaining in position as a vessel implant.

No matter the technique used to open an occluded conduit, e.g., blood vessel, and restore normal fluid flow therethrough, one problem remains: restenosis. A certain percentage of the treated conduits and vessels will reocclude (restenose) after a period of time; occurring in as many as 30-40% of the cases. When restenosis does occur, the original procedure may be repeated or an alternative method may be used to reestablish fluid, e.g., blood, flow.

The relevant commonality shared by each of the above treatment methods is that each one results in some trauma to the conduit wall. Restenosis occurs for a variety of reasons; each involving trauma. Small clots may form on the arterial wall. Small tears in the wall expose the blood to foreign material and proteins which are highly thrombogenic. Resulting clots may grow gradually and may even contain growth hormones released by platelets within the clot. Moreover, growth hormones released by other cells, e.g., macrophages, may cause smooth muscle cells and fibroblasts in the affected region to multiply in an abnormal fashion. There may be an injury in the conduit wall due to the above methods that results in inflammation which may result in the growth of new tissue.

It is known that certain therapeutic substances may have a positive effect on prevention and/or inhibition of restenosis. Several difficulties present themselves in the application of these substances to the affected region in a therapeutic dose. For example, the region in need of treatment is very small and localized. Fluid, e.g., blood, flow in the conduit is continuous, resulting in a flow boundary along the wall which must be disrupted so that the therapeutic substances may reach the localized region of interest within a dose range considered therapeutic. The art fails to adequately provide a mechanism for breaking through this flow boundary to target the region of interest; electing instead generally to place the therapeutic substance into the general flow of the conduit, either by intravenous means or intra-lumen infusion, at a dose that is much higher than therapeutic since the majority of the therapeutic substance will simply flow downstream and either be absorbed systemically or eliminated as waste. For example, intravenous medications are delivered systemically by vein, or regionally, e.g., through intra-lumen infusion without targeting the subject region. Such unnecessary systemic exposure results with unknown and unnecessary adverse results in regions, tissue, and/or organs that are distant from the region of interest. Clearly, systemic delivery and exposure is not well suited to treatment of diseases or conditions having a single intra-lumen region of interest.

The potential utility of localized application of a therapeutic dose of therapeutic substances is not limited to treatment of coronary arteries. Beyond coronary artery delivery, other sites of atherosclerosis, e.g., renal, iliac, femoral, distal leg and carotid arteries, as well as saphenous vein grafts, synthetic grafts and arterio-venous shunts used for hemodialysis would be appropriate biological conduits for a localized therapeutic substance delivery method and mechanism. Nor is the potential utility limited to blood vessels; any biological conduit having a region of interest amenable to treatment may benefit from such a treatment method and mechanism.

The present invention overcomes these deficiencies.

### BRIEF SUMMARY OF THE INVENTION

The invention provides a system, device and method for localized application of therapeutic substances within a biological conduit after the lumen wall has been scored by an eccentric scoring head. One embodiment comprises radial scoring with the eccentric scoring head, with a therapeutic agent coated balloon inflated distal to the scoring and dragged proximally through the scoring. Another embodiment comprises inflation of two anchor balloons on either side of scoring with subsequent inflation of a therapeutic agent coated balloon therebetween which causes the distance between anchor balloons to increase, thus stretching the scoring crevices while applying the agent therein with subsequent closure of crevices on deflation of anchor and application balloons. Another embodiment comprises an inflated anchor balloon with a threaded scoring device wherein the scoring members are coated with agent and rotation of the threaded device enables travel in the proximal direction away from anchor balloon.

In this manner, application of at least one therapeutic dose of the therapeutic substance(s) at the affected region is achieved, while minimizing unwanted systemic exposure and the accompanying undesirable side effects. As a consequence, the need to administer super-therapeutic doses is eliminated.

The figures and the detailed description which follow more particularly exemplify these and other embodiments of the invention.

### BRIEF DESCRIPTION OF THE DRAWINGS

The invention may be more completely understood in consideration of the following detailed description of various embodiments of the invention in connection with the accompanying drawings, which are as follows.

FIG. 1 is a perspective view of one embodiment of a therapeutic agent delivery system comprising an eccentric abrading head of a rotational atherectomy device of the invention;

FIG. 2 is a partial cutaway cross-sectional view of one embodiment of the invention;

FIG. 3 is a partial cutaway cross-sectional view of one embodiment of the invention;

FIG. 4 is a partial cutaway cross-sectional view of one embodiment of the invention;

FIG. 5 is a cutaway cross-sectional view of the indicated portion from FIGS. 3 and 4;

FIG. 6 is a perspective view of one embodiment of a therapeutic agent delivery system comprising an eccentric abrading head of a rotational atherectomy device of the invention;

FIG. 7 is a partial cutaway cross-sectional view of one embodiment of the invention;

FIG. 8 is a partial cutaway cross-sectional view of one embodiment of the invention;

FIG. 9 is a partial cutaway cross-sectional view of one embodiment of the invention;

FIG. 10 is a partial cutaway cross-sectional view of one embodiment of the invention;

FIG. 11 is a partial cutaway cross-sectional view of one embodiment of the invention;

FIG. 12 is a partial cutaway cross-sectional view of one embodiment of the invention;

FIG. 13 is a perspective view of one embodiment of a therapeutic agent delivery system comprising an eccentric abrading head of a rotational atherectomy device of the invention; and

FIG. 14 is a partial cutaway cross-sectional view of one embodiment of the invention.

### DETAILED DESCRIPTION OF THE INVENTION, INCLUDING THE BEST MODE

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

For the purposes of the present invention, the following terms and definitions apply:

“Bodily disorder” refers to any condition that adversely affects the function of the body.

The term “treatment” includes prevention, reduction, delay, stabilization, and/or elimination of a bodily disorder, e.g., a vascular disorder. In certain embodiments, treatment comprises repairing damage cause by the bodily, e.g., vascular, disorder and/or intervention of same, including but not limited to mechanical intervention.

A “therapeutic agent” comprises any substance capable of exerting an effect including, but not limited to therapeutic, prophylactic or diagnostic. Thus, therapeutic agents may comprise anti-inflammatories, anti-infectives, analgesics, anti-proliferatives, and the like including but not limited to antirestenosis drugs. Therapeutic agent further comprises mammalian stem cells. Therapeutic agent as used herein further includes other drugs, genetic materials and biological materials. The genetic materials mean DNA or RNA, including, without limitation, of DNA/RNA encoding a useful protein, intended to be inserted into a human body including viral vectors and non-viral vectors. Viral vectors include adenoviruses, gutted adenoviruses, adeno-associated virus, retroviruses, alpha virus, lentiviruses, herpes simplex virus, ex vivo modified cells (e.g., stem cells, fibroblasts, myoblasts, satellite cells, pericytes, cardiomyocytes, skeletal myocytes, macrophage), replication competent viruses, and hybrid vectors. Non-viral vectors include artificial chromosomes and mini-chromosomes, plasmid DNA vectors, cationic polymers, graft copolymers, neutral polymers PVP, SP1017, lipids or lipoplexes, nanoparticles and microparticles with and without targeting sequences such as the protein transduction domain (PTD). The biological materials include cells, yeasts, bacteria, proteins, peptides, cytokines and hormones. Examples for peptides and proteins include growth factors (FGF, FGF-1, FGF-2, VEGF, Endotherial Mitogenic Growth Factors, and epidermal growth factors, transforming growth factor .alpha. and .beta., platelet derived endothelial growth factor, platelet derived growth factor, tumor necrosis factor .alpha., hepatocyte growth factor and insulin like growth factor), transcription factors, proteinkinases, CD inhibitors, thymidine kinase, and bone morphogenic proteins. These dimeric proteins can be provided as homodimers, heterodimers, or combinations thereof, alone or together with other molecules.

Therapeutic agents further includes cells that can be of human origin (autologous or allogeneic) or from an animal source (xenogeneic), genetically engineered, if desired, to deliver proteins of interest at the transplant site. Cells within the definition of therapeutic agents herein further include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.

Therapeutic agent also includes non-genetic substances, such as: anti-thrombogenic agents such as heparin, heparin derivatives, and urokinase; anti-proliferative agents such as enoxaprin, angiopeptin, or monoclonal antibodies capable of blocking smooth muscle cell proliferation, hirudin, and acetylsalicylic acid, amlodipine and doxazosin; anti-inflammatory agents such as glucocorticoids, betamethasone, dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, methotrexate, azathioprine, adriamycin and mutamycin; endostatin, angiostatin and thymidine kinase inhibitors, taxol and its analogs or derivatives; anesthetic agents such as lidocaine, bupivacaine, and ropivacaine; anti-coagulants such as heparin, antithrombin compounds, platelet receptor antagonists, anti-thrombin anticodies, anti-platelet receptor antibodies, aspirin, dipyridamole, protamine, hirudin, prostaglandin inhibitors, platelet inhibitors and tick antiplatelet peptides; vascular cell growth promotors such as growth factors, Vascular Endothelial Growth Factors, growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vasoactive mechanisms; anti-oxidants, such as probucol; antibiotic agents, such as penicillin, cefoxitin, oxacillin, tobranycin angiogenic substances, such as acidic and basic fibrobrast growth factors, estrogen including estradiol (E2), estriol (E3) and 17-Beta Estradiol; and drugs for heart failure, such as digoxin, beta-blockers, angiotensin-converting enzyme, inhibitors including captopril and enalopril. The biologically active material can be used with (a) biologically non-active material(s) including a solvent, a carrier or an excipient, such as sucrose acetate isobutyrate, ethanol, n-methyl pymolidone, dimethyl sulfoxide, benzyl benxoate and benzyl acetate.

Further, “therapeutic agent” includes, in particular in a preferred therapeutic method of the present invention comprising the administration of at least one therapeutic agent to a procedurally traumatized, e.g., by an angioplasty or atherectomy procedure, mammalian vessel to inhibit restenosis. Preferably, the therapeutic agent is a cytoskeletal inhibitor or a smooth muscle inhibitor, including, for example, taxol and functional analogs, equivalents or derivatives thereof such as taxotere, paclitaxel, abraxane TM, coroxane TM or a cytochalasin, such as cytochalasin B, cytochalasin C, cytochalasin A, cytochalasin D, or analogs or derivatives thereof.

Additional specific examples of “therapeutic agents” that may be applied to a bodily lumen using various embodiments of the present invention comprise, without limitation:
L-Arginine;
Adipose Cells;
Genetically altered cells, e.g., seeding of autologous endothelial cells transfected with the beta-galactosidase gene upon an injured arterial surface;
Erythromycin;
Penicillin:
Heparin;
Aspirin;
Hydrocortisone;
Dexamethasone;
Forskolin;
GP IIb-IIIa inhibitors;
Cyclohexane;
Rho Kinsase Inhibitors;
Rapamycin;
Histamine;
Nitroglycerin;
Vitamin E;
Vitamin C;
Stem Cells;
Growth Hormones;
Hirudin;
Hirulog;
Argatroban;
Vapirprost;
Prostacyclin;
Dextran;
Erythropoietin;
Endothelial Growth Factor;
Epidermal Growth Factor;
Core Binding Factor A;
Vascular Endothelial Growth Factor;
Fibroblast Growth Factors;
Thrombin;
Thrombin inhibitor; and
Glucosamine, among many other therapeutic substances.

The therapeutic agent delivery system of the present invention can be used to apply the therapeutic agent to any surface of a body lumen where a catheter can be inserted. Such body lumen includes, inter alia, blood vessels, urinary tract, coronary vasculature, esophagus, trachea, colon, and biliary tract.

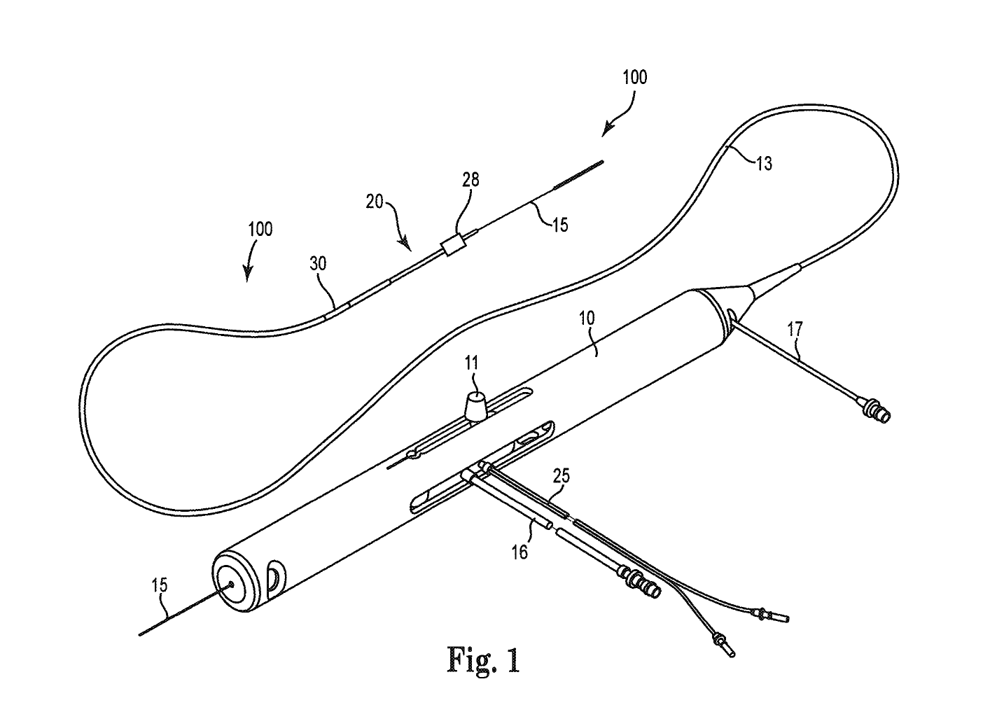

FIG. 1 illustrates one embodiment 100 of a scoring and seeding high-speed rotational atherectomy system of the present invention, elements of which are utilized in various embodiments of the present invention. The device includes a handle portion 10, an elongated, flexible drive shaft 20 having an eccentric scoring head 28 and inflatable balloon 30, inflatable balloon coated 30 with at least one therapeutic agent 37 and disposed proximal to eccentric scoring head 28, and an elongated catheter 13 extending distally from the handle portion 10. The drive shaft 20 is constructed from helically coiled wire as is known in the art and the eccentric scoring head 28 and coated inflatable balloon 30 are fixedly attached thereto. The catheter 13 has a lumen L within which the drive shaft 20, eccentric scoring head 28 and deflated coated inflatable balloon 30 are slidably disposed and further comprises a distal end.

The handle 10 desirably contains a turbine (or similar rotational drive mechanism) for rotating the drive shaft 20 at high speeds. The handle 10 typically may be connected to a power source, such as compressed air delivered through a tube 16. A pair of fiber optic cables 25, alternatively a single fiber optic cable may be used, may also be provided for monitoring the speed of rotation of the turbine and drive shaft 20. Details regarding such handles and associated instrumentation are well known in the industry. The handle 10 also desirably includes a control knob 11 for advancing and retracting the turbine and drive shaft 20 with respect to the catheter 13 and the body of the handle.

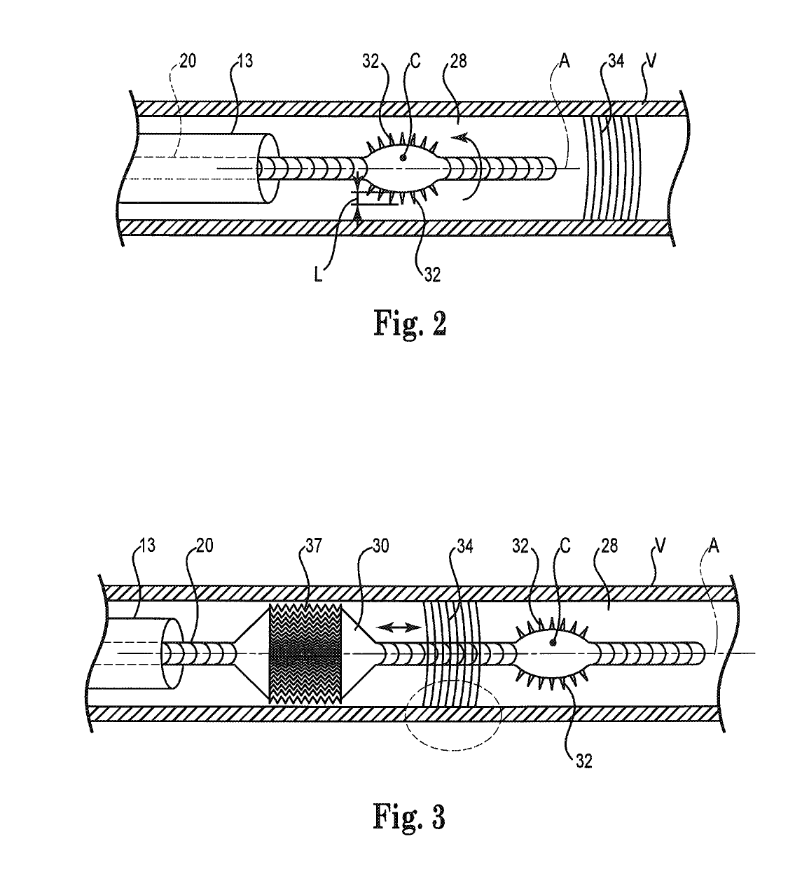

Turning now to FIGS. 2 and 3, the scoring head 28 may comprise at least one scoring element 32 on the external surface(s) of the eccentric scoring head 28 to facilitate scoring of the vessel wall V during high-speed rotation, i.e., 20,000 to 200,000 rpm. Each scoring element 32 comprises a length L, the magnitude of which is a key element to determining the depth of scoring that occurs in operation.

Additional variations of the eccentric scoring head 28 are possible, including an arrangement whereby the wire turns of the drive shaft are enlarged on one side of the drive shaft but not the opposing side, creating an offset of the center of mass C from the axis of rotation A. This arrangement is disclosed within U.S. Pat. No. 6,494,890 to Shturman, the entire contents of which is hereby incorporated herein by reference. The significant part of the eccentric scoring head 28 of the present invention and its various embodiments is that eccentricity is created, i.e., that the center of mass C of the eccentric scoring head 28 is offset from the axis of rotation A of the drive shaft 20. Such eccentricity drives an orbital pattern of rotation for the eccentric scoring head 28 as will be discussed further and which is a significant element of the various embodiments of the present invention.

Accordingly, it should be understood that, as used herein, the word “eccentric” is defined and used herein to refer to either a difference in location between the geometric center of the enlarged abrading head 28 and the rotational axis A of the drive shaft 20, or to a difference in location between the center of mass C of the enlarged abrading head 28 and the rotational axis A of the drive shaft 20. Either such difference, at the proper rotational speeds, will enable the eccentric enlarged abrading head 28 to score walls of vessels having a diameter substantially greater than the nominal, resting diameter of the eccentric scoring head 28. Moreover, for an eccentric scoring head 28 having a shape that is not a regular geometric shape, the concept of “geometric center” can be approximated by locating the mid-point of the longest chord which is drawn through the rotational axis A of the drive shaft 20 and connects two points on a perimeter of a transverse cross-section taken at a position where the perimeter of the eccentric scoring head 28 has its maximum length.

The eccentric scoring head 28 and the scoring elements 32 of the therapeutic agent delivery device of the invention may be constructed of stainless steel, tungsten, titanium or similar material. The eccentric scoring head 28 may be a single piece unitary construction or, alternatively, may be an assembly of two or more abrading head components fitted and fixed together to achieve the objects of the present invention.

As described and illustrated in incorporated reference U.S. Pat. No. 6,494,890, the eccentric scoring head of the present invention comprises a generally spiral orbital path during high-speed rotation and, will create radial scoring throughout the entire circumference of the inner vessel lumen.

Although not wishing to be constrained to any particular theory of operation, applicants believe that offsetting the center of mass from the axis of rotation A produces an “orbital” movement of the eccentric scoring head 28, the diameter of the “orbit” being controllable by varying, inter alia, the rotational speed of the drive shaft 20. Applicants have empirically demonstrated that by varying the rotational speed of the drive shaft 20 one can control the centrifugal force urging the eccentric scoring head 28 against the surface of the stenosis. The centrifugal force can be determined according to the formula:

F_{c}=mΔx(πn/30)²

where F_{c} is the centrifugal force, m is the mass of the eccentric scoring head 28, Δx is the distance between the center of mass of the eccentric scoring head 28 and the rotational axis A of the drive shaft 20, and n is the rotational speed in revolutions per minute (rpm). Controlling this force F_{c}, together with the length L of the individual scoring elements 32 provides control over the depth of scoring in the vessel wall.

Returning to FIGS. 2 and 3, the drive shaft 20 in FIG. 2 is illustrated as extended distally out of catheter 13 lumen to the point that the eccentric scoring head 28 is exposed to the vessel lumen and high-speed rotation of the drive shaft 20 and eccentric scoring head 28 has occurred. Thus, scoring 34 is created in a radial pattern around the circumference of the inner wall of the vessel lumen. The depth of scoring is, as discussed above, controlled by (1) the length L of the scoring elements 32; and (2) by controlling the centrifugal force of the eccentric scoring head 28 during high-speed rotation. At this point in the procedure, inflatable balloon 30 is still retained in a deflated state within the catheter 13 lumen.

FIG. 3 illustrates the drive shaft 20 further extended distally out of the lumen of catheter 13, wherein the eccentric scoring head 28 is disposed distal to the scoring 34 and the inflated coated balloon 30 is shown proximal the scoring 34. No rotation of the drive shaft is occurring while the balloon 30 is inflated. The drive shaft 20 is then advanced distally to scrape the coating comprising at least one therapeutic agent 37 from the balloon and into the radial scoring 34. Translation of the coated balloon, proximally and/or distally across the radial scoring 34 will pull the scoring 34 open, exposing each newly created crevice 36 in the vessel wall during the scoring procedure, smearing the at least one therapeutic agent 37 into the exposed crevice 36 which then closes as the balloon passes the crevice 36 and scoring 34. This arrangement is illustrated in close up in FIG. 5, with scoring 34 shown and crevices 36 filled at least partially with at least one therapeutic agent 37, wherein the crevices 36 are closed as the inflated balloon has passed the scoring 34.

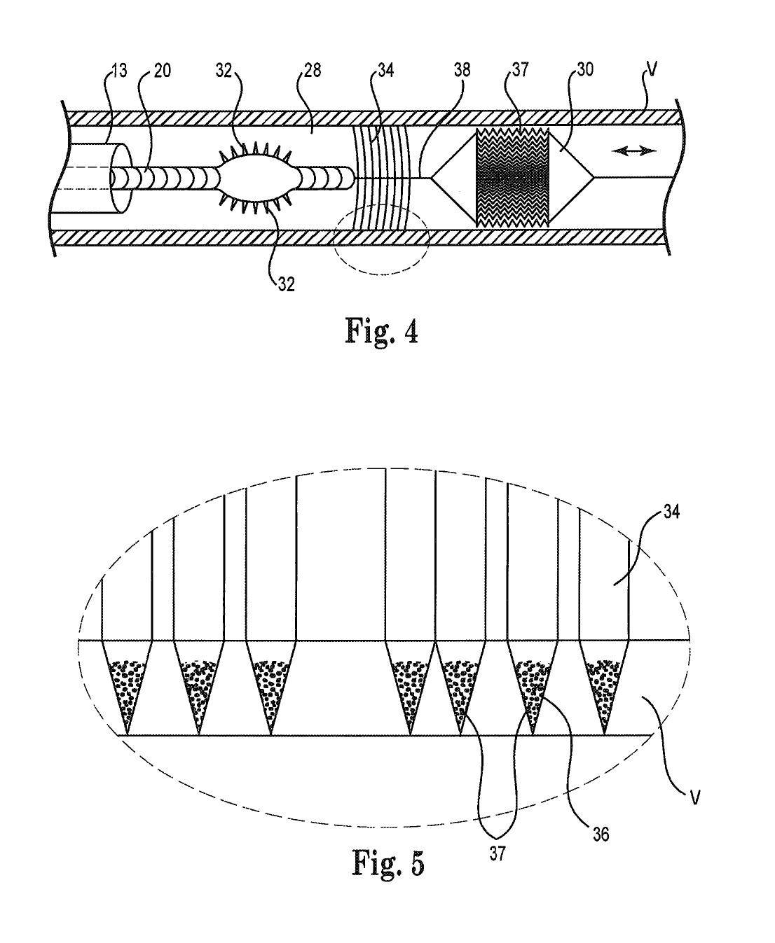

An alternate embodiment is provided in FIG. 4, wherein the inflatable balloon 30, coated with at least one therapeutic agent 37 is slidably disposed in a deflated state within the lumen of drive shaft 20. A wire 38 operatively connects the proximal end of balloon 30 with the handle 10 where an operator may translate the balloon 30 proximally or distally as well as inflate balloon 30 when translated out of the lumen of drive shaft 20. As illustrated, radial scoring 34 is achieved with the high-speed scoring element 28 as described in connection with FIGS. 2 and 3. The deflated but inflatable coated balloon 30 now may be translated distally out of the lumen of the drive shaft 20, where it is inflated at a point distal to the scoring 34. The operator then pulls the wire 38 to translate the inflated coated balloon 30 proximally across the scoring 34, thereby opening the scoring and allowing the therapeutic agent(s) 37 to smear or deposit within the crevices 36 of the scoring 34.

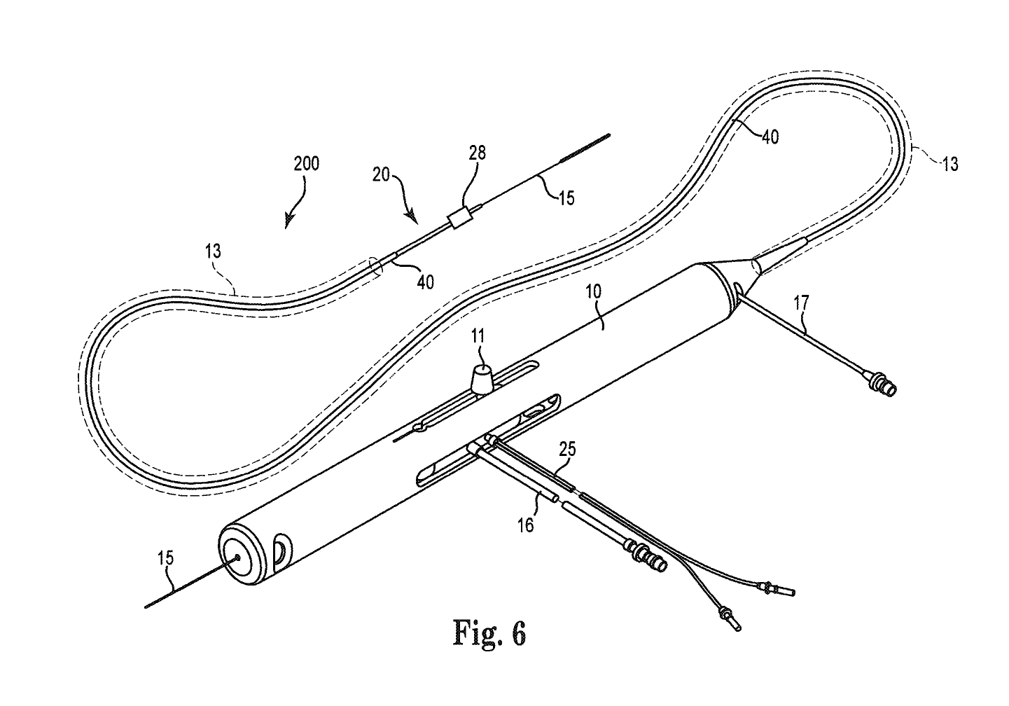

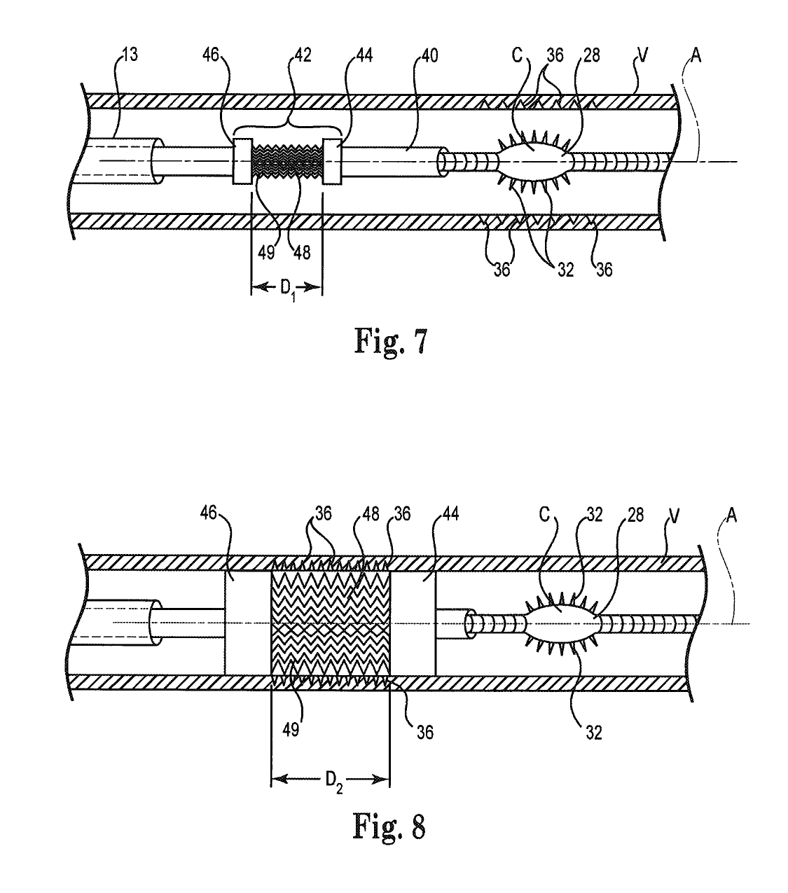

FIG. 6 illustrates a therapeutic delivery system 200 comprising a handle portion 10, an elongated, flexible catheter 13 comprising a lumen therethrough, wherein non-rotatable inflating sheath 40 is slidingly translatably disposed. Sheath 40 comprises a lumen therethrough, within which is slidably and rotatably disposed flexible drive shaft 20, drive shaft 20 having eccentric scoring head 28 attached thereto. 13 Inflating balloon assembly 42 is deflated and slidably disposed within lumen of catheter 13 in FIG. 7. The drive shaft 20 is constructed from helically coiled wire as is known in the art and the eccentric scoring head 28 is fixedly attached thereto.

The handle 10 desirably contains a turbine (or similar rotational drive mechanism) for rotating the drive shaft 20 at high speeds. The handle 10 typically may be connected to a power source, such as compressed air delivered through a tube 16. A pair of fiber optic cables 25, alternatively a single fiber optic cable may be used, may also be provided for monitoring the speed of rotation of the turbine and drive shaft 20. Details regarding such handles and associated instrumentation are well known in the industry. The handle 10 also desirably includes a control knob 11 for advancing and retracting the turbine and drive shaft 20 and may also control axial translation of sheath 40 with respect to the catheter 13 and the body of the handle.

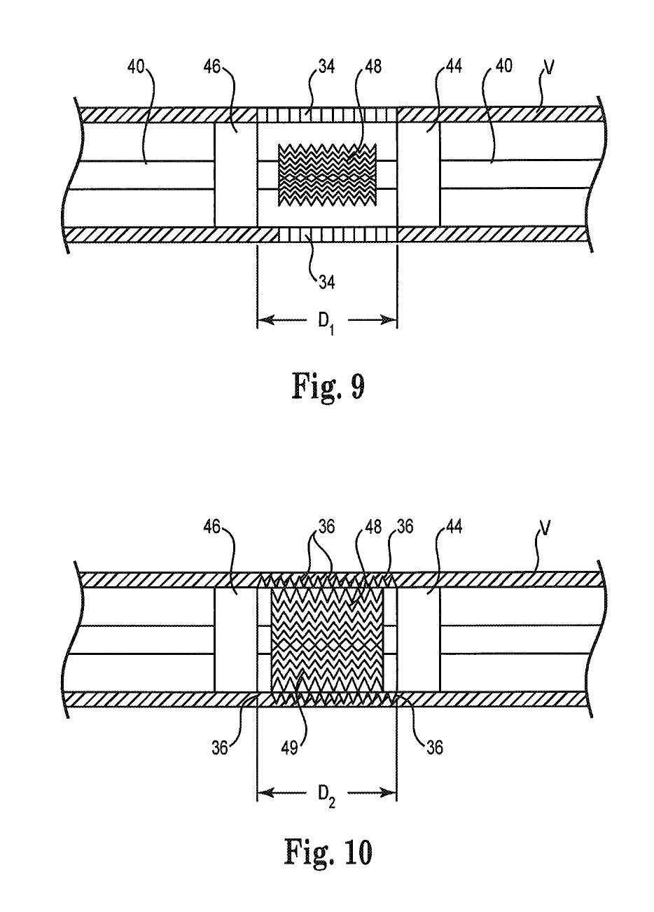

FIGS. 7-10 illustrate the therapeutic delivery system 200 inserted into vessel V, wherein a non-rotatable inflating sheath 40, translatably disposed within the lumen of catheter 13, is distally translated beyond the distal end of the catheter 13. Sheath 40 comprises a lumen, within which the drive shaft 20 is rotatably and slidably disposed. Drive shaft 20 is illustrated as distally translated out of catheter 13, and distally out of the lumen of sheath 40, thereby exposing eccentric scoring head 28 with scoring elements 32 disposed thereon as described supra to the vessel lumen. Non-rotatable inflating sheath 40 comprises an inflating balloon assembly 42, comprising a distal anchor balloon 44, a proximal anchor balloon 46, with a coated balloon 48 disposed therebetween, the coated balloon 48 comprising a coating of at least one therapeutic agent 49. Distal anchor and proximal anchor balloons 44, 46 and coated balloon 48 are deflated until the eccentric scoring head 28 completes its scoring operation, creating crevices 36 in the vessel wall V.

As illustrated in FIGS. 7-10, the distal and proximal anchor balloons 44, 46 are positioned generally distally and proximally to the crevices 36, with the coated balloon 48 disposed therebetween, so that inflation of the coated balloon will engage the scoring 34 created by eccentric scoring head 28. The distal and proximal anchor balloons 44, 46 are first inflated and compressed against the vessel walls V and, as shown in FIG. 7, establishing a first distance D1 therebetween. The coated balloon 48 is then inflated to compression against the proximal and distal balloons 46, 44 as well as the vessel wall V comprising crevices 36. This inflation compression pushes the proximal balloon 46 further proximally and the distal balloon 44 further distally, establishing a second distance D2, wherein D2 is greater than D1. Thus, the crevices 36 are axially stretched open, allowing the coated balloon 48 to pressure its coating of therapeutic agent(s) 49 therein, filling at least partially the stretched open crevices 36. Then, deflation of the coated balloon 48 relaxes the stretched crevices 36, effectively closing the crevices 36 as the distance between the proximal and distal anchor balloons 46, 44 returns to D1. The proximal and distal anchor balloons 46, 44 are then deflated and the system removed. The proximal and distal anchor balloons 46, 44 and coated balloon 48 are inflated with inflation medium as is well known in the art.

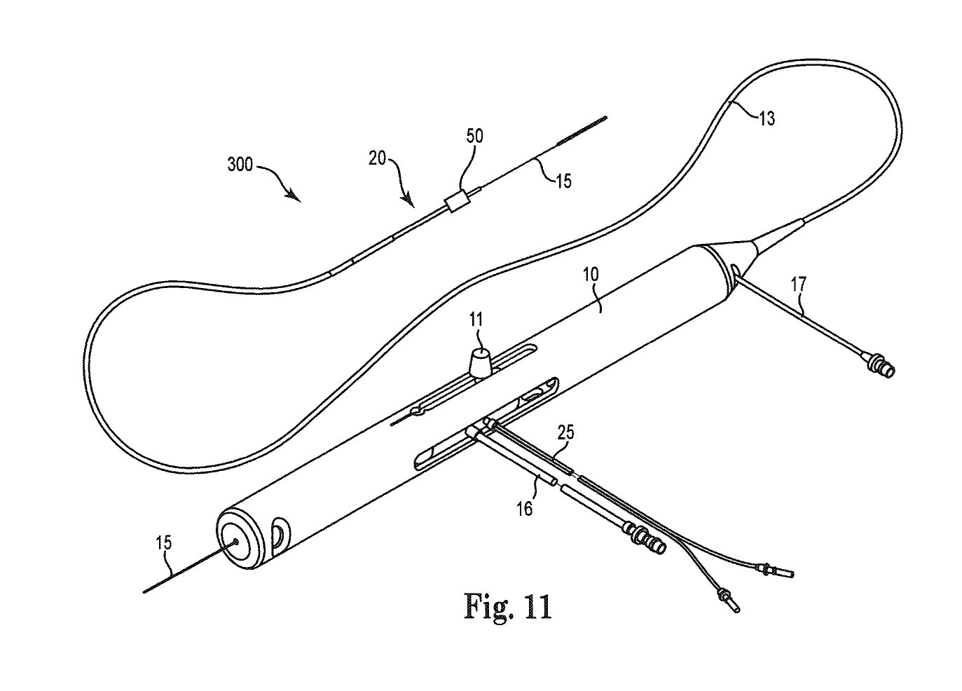

Turning now to FIGS. 11 and 12, illustrates one embodiment 100 of a scoring and seeding high-speed rotational atherectomy system 300 of the present invention, elements of which are utilized in various embodiments of the present invention. The device includes a handle portion 10, an elongated catheter 13 extending distally from the handle portion 10 and having a lumen therethrough, an elongated, flexible drive shaft 20 slidably and rotatably disposed with lumen of catheter 13, the drive shaft 20 comprising a scoring assembly 50 on its distal end. The drive shaft 20 is constructed from helically coiled wire as is known in the art.

The handle 10 desirably contains a turbine (or similar rotational drive mechanism) for rotating the drive shaft 20 at high speeds. The handle 10 typically may be connected to a power source, such as compressed air delivered through a tube 16. A pair of fiber optic cables 25, alternatively a single fiber optic cable may be used, may also be provided for monitoring the speed of rotation of the turbine and drive shaft 20. Details regarding such handles and associated instrumentation are well known in the industry. The handle 10 also desirably includes a control knob 11 for advancing and retracting the turbine and drive shaft 20 with respect to the catheter 13 and the body of the handle.

Scoring assembly 50 comprises a distal inflatable anchor balloon 52 having a proximal end which is fixedly attached to a threaded segment 53. Threaded segment 53 comprises threads thereon and a distal stop 56. Scoring assembly 50 further comprises an inflatable scorer and seeder 54 fixedly attached to the distal end of rotatable drive shaft 20. Inflatable scorer and seeder 54 comprising scoring elements 36 as described supra, with at least one therapeutic agent coated thereon. Alternatively, a reservoir may be provided within scorer and seeder containing therapeutic agent, wherein the scoring elements 36 also comprise a lumen therethrough which is in fluid communication with the scoring element lumen. Still more alternatively, the scoring element 36 may comprise a pre-filled lumen, filled with therapeutic agent. The inflatable scorer and seeder 54 further comprises a threaded distal port 58, within which threaded segment 53 of distal inflatable anchor balloon 52 is threadingly disposed.

In operation, catheter 13, together with drive shaft 20 disposed in lumen of catheter 13, is positioned within patient's lumen adjacent, preferably distally, to the region desired to be scored and seeded. The drive shaft 20 is translated axially and distally until the scoring assembly 50 reached the region of interest. The anchor balloon 52 is then inflated with inflation media using an inflation device as is well known in the art. Inflation of anchor balloon 52 compresses balloon 52 against the lumen wall, fixing balloon 52 in place and preventing rotation thereof. Then, the operator inflates the inflatable scorer and seeder 54 and actuates the drive shaft 20, causing it to rotate. As this rotation progresses, several things occur. The scoring elements 36 begin to score the lumen wall V and in the various embodiments, the therapeutic agent(s) is deposited within the scoring. Rotation of the drive shaft 20 results in concurrent rotation of the inflatable scorer and seeder 54, in particular counterclockwise rotation of inflatable scorer and seeder 54 results in proximal threaded movement of the scorer and seeder 54 as the threaded distal port 58 engages the threads of threaded segment 53. As this proximal threading movement occurs, the scoring elements 36 also score proximally in the vessel wall V, leaving the therapeutic agent(s) within the scoring. The rotation of scorer and seeder 54 may progress until the distal stop 56 is encountered, which stops the proximal threaded translational movement of scorer and seeder 54. The anchor balloon 52 and the scorer and seeder 54 are deflated, withdrawn proximally into lumen of catheter 13 and removed from the patient's lumen.

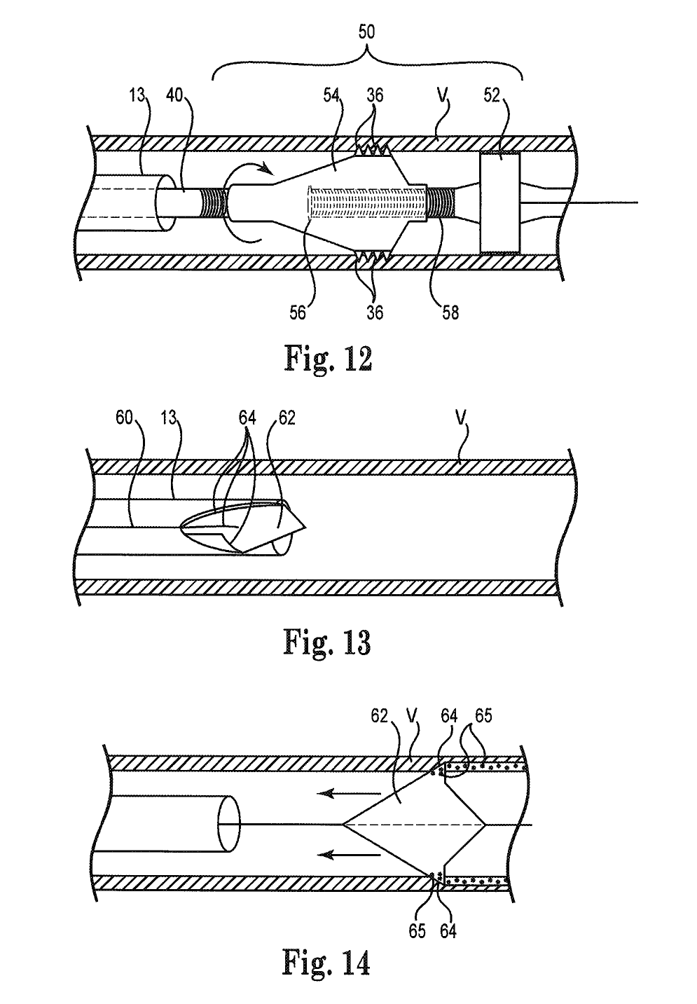

With reference now to FIGS. 13 and 14, a catheter 13 is provided with a lumen therein, catheter 13 is inserted to the region of interest in the vessel. A slicer 62 is disposed within lumen of catheter 13 in a first retracted position. At least one, but preferably two or more, fins 64 are provided on the body of slider 62 as illustrated. The fins 64 are, in the slicer's first retracted position, retracted to allow axial translation within lumen of catheter 13. A wire 60 is attached to the proximal end of slicer 62, whereby proximal and/or distal translation of slicer 62 is achieved. Once the catheter 13 is positioned in the vessel, the operator translates the slicer 62 out of the lumen of the catheter 13, whereby slicer 62 achieves automatically its second, expanded position as in FIG. 12. In this second, expanded position, fins 64, automatically expand, slicing into the vessel wall V. Fins 64 may be coated with at least one therapeutic agent 65, so that frictional contact with the vessel wall V during slicing into wall V will release some of the at least one therapeutic agent 65 into wall V. Proximally translation of slicer in its second, expanded position by pulling on wire 60 will cause the coated fins 64 to slice proximally through the vessel wall V, leaving a coating of the at least one therapeutic agent therein. Once the slicer 62 reaches the lumen of the catheter, it is forced into the first, retracted position as it translates proximally therein for removal from the vessel along with catheter 13.

The present invention should not be considered limited to the particular examples described above, but rather should be understood to cover all aspects of the invention. Various modifications, equivalent processes, as well as numerous structures to which the present invention may be applicable will be readily apparent to those of skill in the art to which the present invention is directed upon review of the present specification.

What is claimed is:

1. A high-speed rotational atherectomy system for delivering at least one therapeutic agent to a vessel wall, comprising:
a guide wire having a maximum diameter less than the diameter of the vessel;
a flexible elongated, rotatable drive shaft advanceable over the guide wire, the drive shaft having a rotational axis;
an eccentric scoring head comprising an external surface and attached to the drive shaft, the eccentric scoring head comprising an external surface and at least one scoring element on the external surface, and a center of mass that is offset from the rotational axis of the drive shaft; and
an inflatable balloon attached to the drive shaft, the inflatable balloon coated with at least one therapeutic agent and positioned proximally on the drive shaft from the eccentric scoring head,
wherein the eccentric scoring head scores the vessel wall, creating crevices therein and the inflated balloon is translated axially proximally and/or distally through the crevices and smears its coating into the crevices.

2. A high-speed rotational atherectomy system for delivering at least one therapeutic agent to a vessel wall, comprising:
a guide wire having a maximum diameter less than the diameter of the vessel;
a flexible elongated, rotatable drive shaft advanceable over the guide wire, the drive shaft having a rotational axis;
an eccentric scoring head comprising an external surface and attached to the drive shaft, the eccentric scoring head comprising an external surface and at least one scoring element on the external surface, and a center of mass that is offset from the rotational axis of the drive shaft; and
an inflatable balloon attached to a wire and disposed within the lumen of the drive shaft, the inflatable balloon coated with at least one therapeutic agent and positioned distally on the drive shaft from the eccentric scoring head,
wherein the eccentric scoring head scores the vessel wall, creating crevices therein and the deflated balloon is translated axially distally through the crevices, inflated and then pulled proximally by the wire through the crevices where it smears its coating into the crevices.

3. A method for delivering at least one therapeutic agent to a vessel wall, comprising:
providing a guide wire having a maximum diameter less than the diameter of the vessel;
providing a flexible elongated, rotatable drive shaft advanceable over the guide wire, the drive shaft having a rotational axis;
providing an eccentric scoring head comprising an external surface and attached to the drive shaft, the eccentric scoring head comprising an external surface and at least one scoring element on the external surface, and a center of mass that is offset from the rotational axis of the drive shaft;
providing an inflatable balloon attached to the drive shaft, the inflatable balloon coated with at least one therapeutic agent and positioned proximally on the drive shaft from the eccentric scoring head;
radially scoring the vessel wall with the eccentric scoring head;
creating crevices in the vessel wall with the radial scoring;
translating the inflated balloon axially proximally and/or distally against the crevices;
opening the crevices with the inflated balloon;
smearing the balloon's coating of at least one therapeutic agent into the opened crevices;
deflating the inflatable balloon; and
closing the crevices.

4. A high-speed rotational atherectomy system for delivering at least one therapeutic agent to a biological lumen wall during a high-speed rotation atherectomy procedure within the biological lumen, comprising:
a guide wire having a maximum diameter less than the diameter of the biological lumen;
a flexible elongated, rotatable drive shaft advanceable over the guide wire, the drive shaft having a rotational axis;
an eccentric scoring head comprising an external surface and attached to the drive shaft, the eccentric scoring head comprising an external surface and at least one scoring element on the external surface, and a center of mass that is offset from the rotational axis of the drive shaft;
a non-rotating inflating sheath comprising a lumen within which the drive shaft is slidably and rotatably disposed; and
an inflating balloon assembly disposed on the non-rotating inflating sheath comprising an inflatable proximal anchor balloon, an inflatable distal anchor balloon, and an inflatable coated balloon comprising a coating of at least one therapeutic agent and disposed between the proximal and distal anchor balloons, wherein the inflating balloon assembly comprises a first distance between the inflatable proximal and distal anchor balloons when the coated balloon is deflated and a second distance when the between the inflatable proximal and distal anchor balloons when the coated balloon is inflated.

5. The system of claim 4, wherein the eccentric scoring head creates radial scoring comprising crevices on the lumen wall, and wherein the deflated coated balloon is positioned in the scoring proximate the crevices.

6. The system of claim 5, wherein the proximal anchor and distal anchor balloons are inflated, thereby achieving the first distance therebetween.

7. The system of claim 6, wherein the coated balloon is inflated, thereby achieving the second distance between the proximal and distal anchor balloons, stretching the crevices open, compressing the at least one therapeutic agent into the opened crevices.

8. The system of claim 7, wherein the coating balloon and proximal and distal anchor balloons are deflated.

9. A method for applying at least one therapeutic agent to a vessel wall, comprising:
providing a guide wire having a maximum diameter less than the diameter of the vessel;
providing a flexible elongated, rotatable drive shaft advanceable over the guide wire, the drive shaft having a rotational axis;
providing an eccentric scoring head comprising an external surface and attached to the drive shaft, the eccentric scoring head comprising an external surface and at least one scoring element on the external surface, and a center of mass that is offset from the rotational axis of the drive shaft;
providing a non-rotating inflating sheath comprising a lumen within which the drive shaft is slidably and rotatably disposed; and
providing an inflating balloon assembly disposed on the non-rotating inflating sheath comprising an inflatable proximal anchor balloon, an inflatable distal anchor balloon, and an inflatable coated balloon comprising a coating of at least one therapeutic agent and disposed between the proximal and distal anchor balloons, wherein the inflating balloon assembly comprises a first distance between the inflatable proximal and distal anchor balloons when the coated balloon is deflated and a second distance when the between the inflatable proximal and distal anchor balloons when the coated balloon is inflated.
radially scoring the lumen wall and creating crevices thereby;
positioning the proximal anchor balloon proximal to the radial scoring;
positioning the distal anchor balloon proximal to the radial scoring;
positioning the coated balloon proximate the crevices;
inflating the proximal and distal anchor balloons;
establishing a first distance between the proximal and distal anchor balloons;
inflating the coated balloon;
establishing a second distance between the proximal and distal anchor balloons, wherein the second distance is greater than the first distance;
stretching the crevices open;
compressing the at least one therapeutic agent coated on the coated balloon into the opened crevices;
deflating the coated balloon;
establishing the first distance between the proximal and distal anchor balloons;
closing the crevices; and
deflating the proximal and distal anchor balloons.

10. A rotational atherectomy system for delivering at least one therapeutic agent to a biological lumen wall, comprising:
a guide wire having a maximum diameter less than the diameter of the biological lumen;
a flexible elongated, rotatable drive shaft advanceable over the guide wire and comprising a distal end;
a scoring assembly disposed on the distal end of the drive shaft, comprising
a distal inflatable anchor balloon having a proximal end which is fixedly attached to a threaded segment, threaded segment comprising threads thereon and a distal stop;
an inflatable scorer and seeder fixedly attached to the distal end of rotatable drive shaft and comprising at least one scoring element thereon, comprising a threaded distal port comprising threads, the threaded segment threadingly disposed within threaded distal port,
wherein counterclockwise rotation of the inflatable scorer and seeder threads the inflatable scorer and seeder proximally on threaded segment.

11. The system of claim 10, wherein the distal stop stops the proximal threading of the inflatable scorer on threaded segment.

12. The system of claim 11, wherein the at least one scoring element comprises a coating of at least one therapeutic agent thereon.

13. The system of claim 10, wherein the at least one scoring element comprises a lumen therethrough and in fluid communication with a reservoir of at least one therapeutic agent.

14. A method for delivering at least one therapeutic agent to a vessel wall, comprising:
providing a guide wire having a maximum diameter less than the diameter of the vessel;
providing a flexible elongated, rotatable drive shaft advanceable over the guide wire and comprising a distal end;
providing a scoring assembly disposed on the distal end of the drive shaft, comprising a distal inflatable anchor balloon having a proximal end which is fixedly attached to a threaded segment, threaded segment comprising threads thereon and a distal stop, and
an inflatable scorer and seeder fixedly attached to the distal end of rotatable drive shaft and comprising at least one scoring element thereon wherein the at least one scoring element is coated with at least one therapeutic agent, comprising a threaded distal port comprising threads, the threaded segment threadingly disposed within threaded distal port,
wherein counterclockwise rotation of the inflatable scorer and seeder threads the inflatable scorer and seeder proximally on threaded segment;
inflating the distal inflatable anchor balloon;
rotating the inflatable scorer and seeder counterclockwise;
proximally threading inflatable scorer and seeder over threaded segment;
scoring the vessel wall with the at least scoring element coated with at least one therapeutic agent;
delivering at least one therapeutic agent into the scoring; and
stopping the proximal threading of the inflatable scorer over threaded segment with distal stop.

15. A system for delivering at least one therapeutic agent to the wall of a biological conduit, comprising:
an elongated, flexible catheter comprising a lumen therethrough;
a slicer comprising at least one fin, the fin coated with at least one therapeutic agent, and a proximal end, the slicer capable of achieving a first, retracted position for axial translation through the lumen of the catheter, and a second, expanded position when released from the lumen of the catheter; and
a wire attached to the proximal end of the slicer, whereby the slicer may be translated axially,
wherein the at least one fin of the slicer in the second, expanded position slices into the wall of conduit and wherein the slicer may be translated proximally with wire.
